(19) United States
(12) Patent Application Publication
(10) Pub. No.: US 2005/0256503 A1
(43) Pub. Date: Nov. 17, 2005

(54) Tapered catheter delivery system

(75) Inventor: Jeffrey A. Hall, Hoover, AL (US)

Correspondence Address: JASON R. KRAUS;FAEGRE & BENSON, LLP, 2200 WELLS FARGO CENTER, 90 SOUTH SEVENTH STREET, MINNEAPOLIS, MN, 55402-3901, US

(73) Assignee: Cardiac Pacemakers, Inc., St. Paul, MN (US)

(21) Appl. No.: 11/188,566

(22) Filed: Jul. 25, 2005

Related U.S. Application Data
(63) Continuation-in-part of application No. 10/140,540, filed on May 7, 2002, now Pat. No. 6,939,327.
(60) Provisional application No. 60/677,114, filed on May 3, 2005.

(51) Int. Cl. A61M 25/00

(52) U.S. Cl. 604/523

(57) ABSTRACT

Various catheters and catheter systems, along with methods for using such are described. In some cases, the catheters and catheter systems exhibit a tapered distal end, with the taper formed integral to a catheter sheath and/or through the interaction of two catheter sheaths.

### CROSS-REFERENCE TO RELATED APPLICATION(S)

This application claims priority under section 119(e) to U.S. provisional patent application 60/677,114, filed May 3, 2005, which is hereby incorporated by reference. This application is a continuation-in-part of U.S. patent application Ser. No. 10/140,540 entitled “PEEL-AWAY SHEATH,” and filed by Hall et al. on May 7, 2002, the entirety of which is incorporated herein by reference.

### BACKGROUND

Embodiments of the present invention relate generally to delivery systems for intervention devices. More particularly, embodiments of the present invention relate to tapered end catheters, elements for forming tapered end catheters, and/or methods for using such.

Various medical procedures involve percutaneous introduction of an intervention device into a body via the lumen of an artery using a catheter. In some cases, advancing the catheter to a work site within the body is either difficult or impossible. This can be caused by a number of factors including, but not limited to, vascular restriction, and can result in a sub-optimum deployment of an intervention device.

Hence, for at least the aforementioned reasons, there exists a need in the art for advanced systems and methods for deploying intervention devices.

### SUMMARY

Embodiments of the present invention provide various systems and methods that can be utilized in deploying intervention devices and/or for other applications involving catheterization. Various embodiments of the present invention provide catheters with a tapered distal end. In some cases, the tapered distal end is formed by tapering the distal end of a single elongate tube, while in other cases, the tapered distal end is formed by the interaction of two or more elongate tubes.

One embodiment of the present invention provides a catheter sheath for forming a tapered distal end of a catheter system. The catheter sheath includes an elongate tube having a proximal end, a distal end, and a lumen extending between the proximal end and the distal end. The elongate tube is disposable over a guide catheter and within an outer elongate tube such that the interaction of the distal end of the elongate tube and the distal end of the outer elongate tube form a stepped taper. In some instances, the elongate tube includes an opening extending from the proximal end to the first distal end. The opening allows for wrapping the elongate tube around an element disposed within the outer elongate tube. In one particular case, the element disposed within the outer elongate tube is a guide catheter.

In other embodiments of the present invention, catheter systems are provided that include at least one elongate tube having a proximal end, a tapered distal end, and a lumen extending between the proximal end and the distal end. In some cases, the at least one elongate tube includes an inner elongate tube and an outer elongate tube. Each of the inner and outer elongate tubes includes proximal and distal ends with a lumen extending between the proximal and distal ends. In such cases, the aforementioned tapered distal end can be formed by extending the inner elongate tube through the outer elongate tube and beyond the distal end of the elongate tube to form a stepped taper. In particular cases, the inner elongate tube can include an opening or slit extending from the proximal end to the distal end. This opening allows the inner elongate tube to wrap around an element previously disposed within the outer elongate tube. In one particular case, the previously disposed element is a guide catheter.

In some cases, the taper of the catheter system is a smooth taper, while in other cases the taper is a rounded step taper. Such rounded step and smooth tapers can be concave in or concave out. In various instances, the distal end is formed of a deformable material which, one or more cases can have memory. Such memory can, for example, cause the distal end to return to a prior shape, or some approximation of the prior shape when a deforming force is removed therefrom.

In various cases, a guide having an outer diameter that is greater than the inner diameter of the elongate tube lumen can be disposed within the elongate tube lumen. The guide can be advanced within the elongate tube lumen until the distal end of the guide is coextensive with the distal end of the elongate tube. In such a configuration, the distal end or tip of the elongate tube deforms. In some cases where the tip is made of a material having memory, the distal end reforms when the guide is retracted within the elongate tube lumen.

In one or more instances, the distal end is formed of two or more materials with one material forming the farthest end, and another material forming a portion of the elongate tube abutting the material forming the farthest end. Further, in some cases, the tapered distal end can exhibit a constant inner lumen diameter, while the outer diameter is tapered. Alternatively, in some cases, both the inner lumen diameter and the outer diameter of the distal end are tapered.

Yet other embodiments of the present invention provide a catheter kit, wherein the catheter kit includes a guide and a catheter sheath. The guide includes a guide elongate tube having a guide distal end, a guide proximal end, and a guide lumen extending from the guide distal end to the guide proximal end. The catheter sheath includes at least one elongate tube having a tapered catheter distal end, a catheter proximal end, and a catheter lumen extending from the catheter distal end to the catheter proximal end.

Further embodiments of the present invention provide a method for disposing an object in the coronary sinus of a heart. Such methods include, providing a catheter sheath that includes at least one elongate tube. The at least one elongate tube includes a tapered distal end, a proximal end, and a lumen extending between the distal and proximal ends. In some cases, the at least one elongate tube includes an outer elongate tube and an inner elongate tube, and the methods further comprise inserting the distal end of the outer elongate tube within the right atrium of a heart, and thereafter inserting the distal end of the inner elongate tube through the lumen of the outer elongate tube and extending beyond the distal end of the outer elongate tube. In such a situation, the combination of the distal end of the outer elongate tube and that of the inner elongate tube forms a stepped taper. A guide can also be provided that includes a distal end and a proximal end. In such a case, the methods can include inserting the guide into the heart such that it contacts the coronary sinus (or opening thereof) of the heart, and sliding the catheter sheath over the guide such that the distal end of the catheter sheath contacts the coronary sinus (or opening thereof). In addition, the distal end of the catheter sheath is advanced into the coronary sinus.

Yet further embodiments of the present invention provide methods of manufacturing a catheter sheath. The methods include providing a cylindrical elongate tube that includes a lumen extending from a proximal end of the elongate tube to a distal end of the elongate tube, and providing a tip tube that includes a lumen extending from a proximal end of the tip tube to a distal end of the tip tube. The distal end of the elongate tube is attached to the proximal end of the tip tube such that an outer diameter of the proximal end of the tip tube is greater than an outer diameter of the distal end of the tip tube. In some cases, the distal diameter of the tip tube being less than the proximal end of the tip tube is defined prior to attaching the tip tube to the elongate tube. In other cases, prior to attaching the tip tube, the outer diameter of the proximal end of the tip tube is approximately the same as the outer diameter of the distal end, and after or during attachment, the differences in the diameters is defined.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 depicts the distal end of a nested catheter;

FIG. 2a depicts the distal end of a nested catheter with a stepped, tapered tip formed though use of an intermediate catheter sheath in accordance with some embodiments of the present invention;

FIG. 2b depicts a multi-material distal end of an intermediate catheter sheath in accordance with one or more embodiments of the present invention;

FIGS. 2c-2f illustrate steps for post insertion installation of the intermediate catheter sheath of FIG. 2a in accordance with various embodiments of the present invention;

FIG. 3a illustrates the distal end of a concave in, tapered catheter in accordance with some embodiments of the present invention;

FIG. 3b illustrates the distal end of a concave in, tapered catheter of FIG. 3 where the catheter tip deformable in accordance with various embodiments of the present invention;

FIG. 4 illustrates the distal end of a smooth tapered catheter in accordance with one or more embodiments of the present invention;

FIG. 5 illustrates a human heart with a catheter disposed therein;

FIG. 6 illustrates what is being referred to as a “coronary sinus spasm” which is a restriction to which various systems and methods of the present invention can be applied; and

FIGS. 7 depict catheter kits in accordance with various embodiments of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

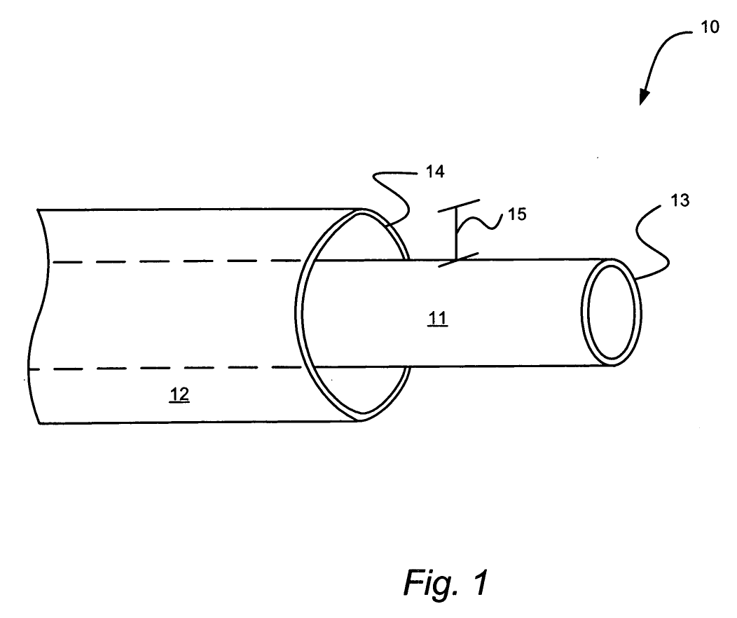

Various catheterization procedures can use a nested catheter system 10 as depicted in FIG. 1. Such procedures can involve directing the catheter system 10, which includes a guide catheter 11 disposed within an outer sheath 12, to a work site. The guide catheter 11 and the outer sheath 12 have respective outer diameters 13, 14 with a tolerance 15 between the catheters. With the catheter system 10 in place at a work site, an intervention device can be deployed through a lumen of the guide catheter 11 and positioned at the work site. The catheter system 10 can then be removed, leaving the intervention device in place. In some cases, however, the guide catheter 11 can be routed to the work site, but the progress of the outer sheath 12 is impeded and incapable of reaching the work site. In such cases, one or more catheters in accordance with the present invention can be used to advance a catheter system beyond the obstruction.

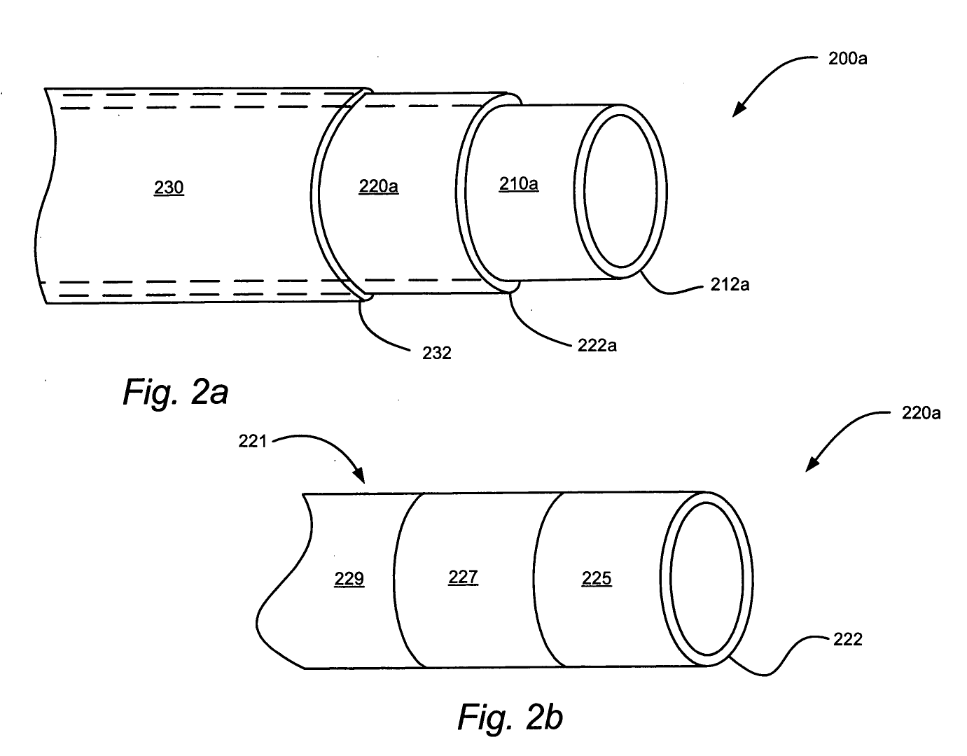

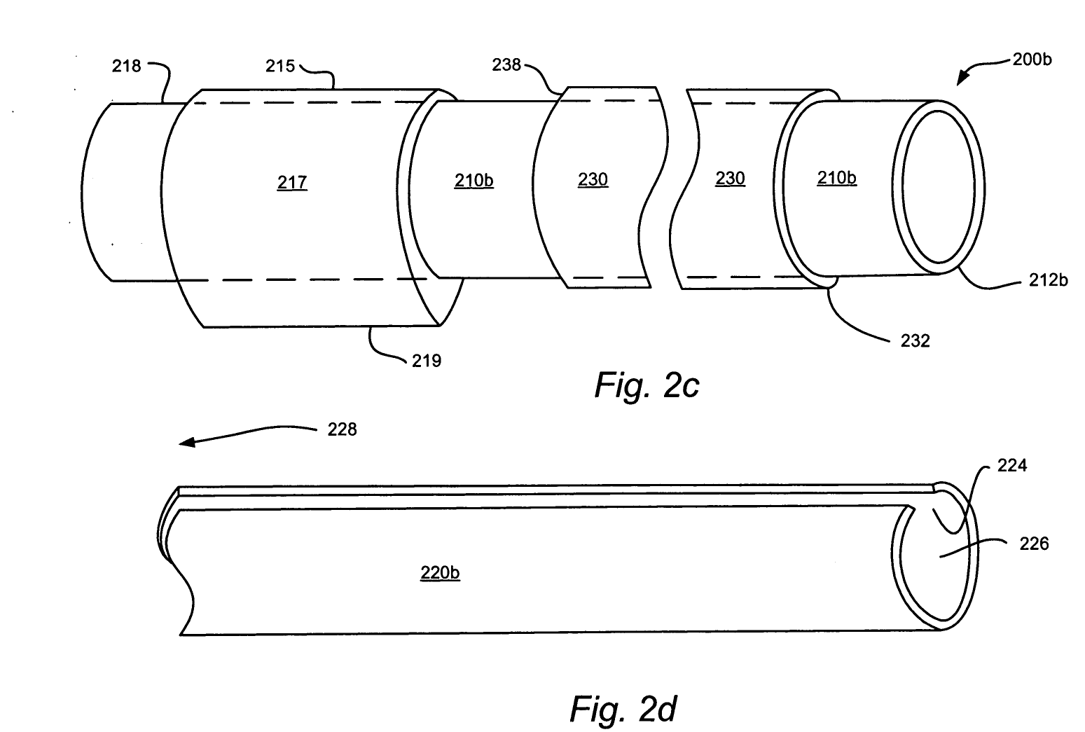

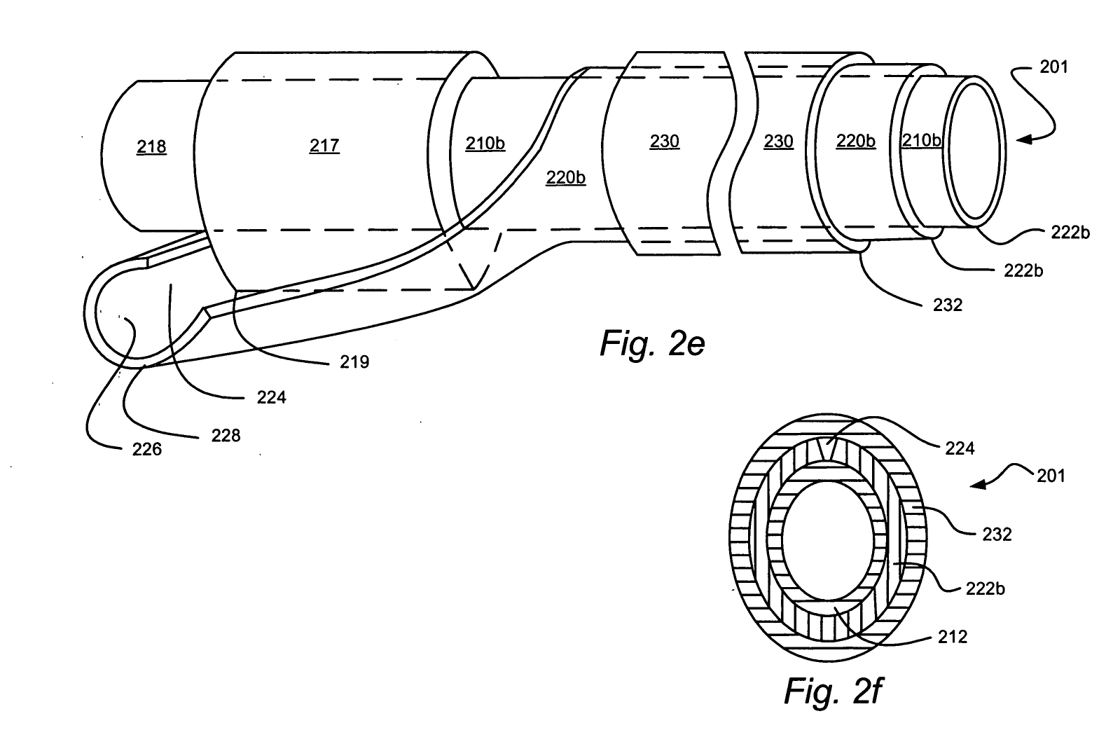

FIG. 2a shows the distal end of a nested catheter system 200 with a stepped or tapered tip formed though use of an intermediate catheter sheath, according to some embodiments of the present invention. The nested catheter system 200 includes a guide catheter 210 having a distal end 212, an intermediate catheter 220 having a distal end 222, and an outer catheter sheath 230 having a distal end 232. The nested catheter system 200 includes a stepped or tapered tip with a taper angle defined by the interaction of the ends of the outer catheter sheath 230 and the intermediate catheter sheath 220. The taper angle can be adjusted by moving the distal end 222 relative to the distal end 232.

A variety of materials can be used to form the various portions of the catheter system 200. For example, one or more of the portions can be made of a polymer possessing a high modulus of elasticity, such as polyetheretherketone (PEEK). Alternatively, or in addition, one or more of the portions can be made of a flexible, intermediate-durometer polymer such as polyether block amide, known commercially as Pebax™. Other polymers including, but not limited to other extrudable polymers like FEP, and the like can also be used.

In some embodiments, the inner diameter of the distal end 232 is approximately 2.21 millimeters, and the outer diameter is approximately 2.67 millimeters. In these embodiments, the outer diameter of the distal end 212 is approximately 2.06 millimeters, and the inner diameter is approximately 1.63 millimeters. Following the illustrated embodiment in light of the aforementioned measurements, the outer diameter of the distal end 222 is less than 2.21 millimeters, and the inner diameter is greater than 2.06 millimeters. In one particular embodiment, the outer diameter of the distal end 222 is approximately 2.19 millimeters and the inner diameter is approximately 2.08 millimeters.

In another embodiment, the inner diameter of the distal end 232 is approximately 2.5 millimeters, and the outer diameter is approximately 2.9 millimeters. In these embodiments, the outer diameter of the distal end 212 is approximately 2.0 millimeters, and the inner diameter is approximately 1.5 millimeters. Following the illustrated embodiment in light of the aforementioned measurements, the outer diameter of the distal end 222 is less than 2.5 millimeters, and the inner diameter is greater than 2.0 millimeters. In one particular embodiment, the outer diameter of the distal end 222 is approximately 2.42 millimeters and the inner diameter is approximately 2.08 millimeters.

In some cases, the tip of the intermediate catheter sheath 220 is formed of a material that is more rigid than that of the guide catheter 210, while in other cases the tip of the intermediate catheter sheath 220 is formed of a material that is less rigid or of equal rigidity than that of the guide catheter 210. Further, in some cases, the tip of the intermediate catheter sheath 220 is formed of a material that is more rigid than that of the outer catheter sheath 230, while in other cases the tip of the intermediate catheter sheath 220 is formed of a material that is less rigid or of equal rigidity than that of the outer catheter sheath 230.

One particular embodiment of the present invention is illustrated in FIG. 2b, which shows an expanded view of a tip 221 of the intermediate catheter sheath 220. As illustrated, the tip 221 includes three different regions 225, 227, 229. Each of the regions 225, 227, 229 is formed of different materials with the region 225 being the most pliable material at a given temperature and the region 229 being the least pliable material at the same temperature. The region 227 is an intermediate region formed of a material capable of acting as a joint between the outer regions 225, 229. It should be noted that fewer or more than the illustrated number of regions can be used in accordance with the present invention.

In one particular embodiment, each of the regions 225, 227, 229 are formed of a bio-compatible polymer material as is known in the art. Further, in various cases, the bio-compatible polymer material is selected based on the material used to make the guide catheter 210 and/or the outer catheter sheath 230. In particular, it may be desirable for the intermediate catheter sheath 220 to slide easily over the guide catheter 210 and/or easily within the outer catheter sheath 230 where tolerances between such elements become tight. Thus, in some cases, a coating such as PTFE or the like can be included with one or more of the guide catheter 210, the intermediate catheter sheath 220 and/or the outer catheter sheath 230. Based on the disclosure provided herein, one of ordinary skill in the art will appreciate other materials and/or coatings that can be used in accordance with the present invention.

FIG. 2c illustrates another embodiment of the catheter system 200 prior to installation of the intermediate catheter sheath 220. As depicted in FIG. 2c, the catheter system 200b includes a guide catheter 210b with an assembly accessory 217 that is useful for inserting the intermediate catheter sheath 220b depicted in FIG. 2d over guide catheter 210b and within outer catheter sheath 230. In some cases, assembly accessory 217 is affixed to a proximal portion 218 of guide catheter 210b. Assembly accessory 217 can include a rounded portion 215 positioned around a proximal portion of guide catheter 210b, and a relatively pointed portion 219 extending away from guide catheter 210b.

As depicted in FIG. 2d, an intermediate catheter sheath 220b includes a slit or opening 224 extending from a distal end 226 of intermediate catheter sheath 220b to a proximal end 228 thereof. In some embodiments of the present invention, the intermediate catheter sheath 220 can be a continuous elongate tube with an opening on one or both ends of the tube, while in other cases the intermediate catheter sheath 220 can be a slit tube such as that illustrated in FIGS. 2d-2f. In one embodiment, the distal end 226 is beveled or tapered. This feature can facilitate passage through a venous restriction by the user twisting the intermediate sheath 220 to vary the location of the beveled tip with respect to the restriction. In another embodiment, the intermediate sheath 220 includes one or more electrodes 223. These electrodes 223 may be sputtered onto the intermediate sheath 220, which will enable use of the sheath 220 for measuring interatrial conduction times and hemodynamic optimization.

As illustrated in FIG. 2e, to assemble intermediate catheter sheath 220b with catheter system 200b, distal end 226 of intermediate catheter sheath 220b is placed against the relatively pointed portion 219 of assembly accessory 217 such that relatively pointed portion 219 is positioned within opening 224. Intermediate catheter sheath 220 is then advanced along over assembly accessory 217, along and over guide catheter 210b, and within outer catheter sheath 230. As intermediate catheter sheath 220b is advanced, distal end 222b comes into proximity of one or more of distal ends 212, 232. In this position, a distal end 201 of catheter system 200b appears as illustrated in FIGS. 2e-2f, and can include a stepped, tapered tip with a taper angle defined by the interaction of the tips of outer catheter sheath 230 and that of intermediate catheter sheath 220b. The straight, longitudinal pattern of opening 224 can be replaced by a helical pattern as more fully discussed in U.S. patent application Ser. No. 10/140,540, which was previously incorporated herein by reference for all purposes. In another embodiment, the system 200 does not include the inner guide catheter 210. In this embodiment, a lead may be introduced through a lumen in the intermediate sheath 220b.

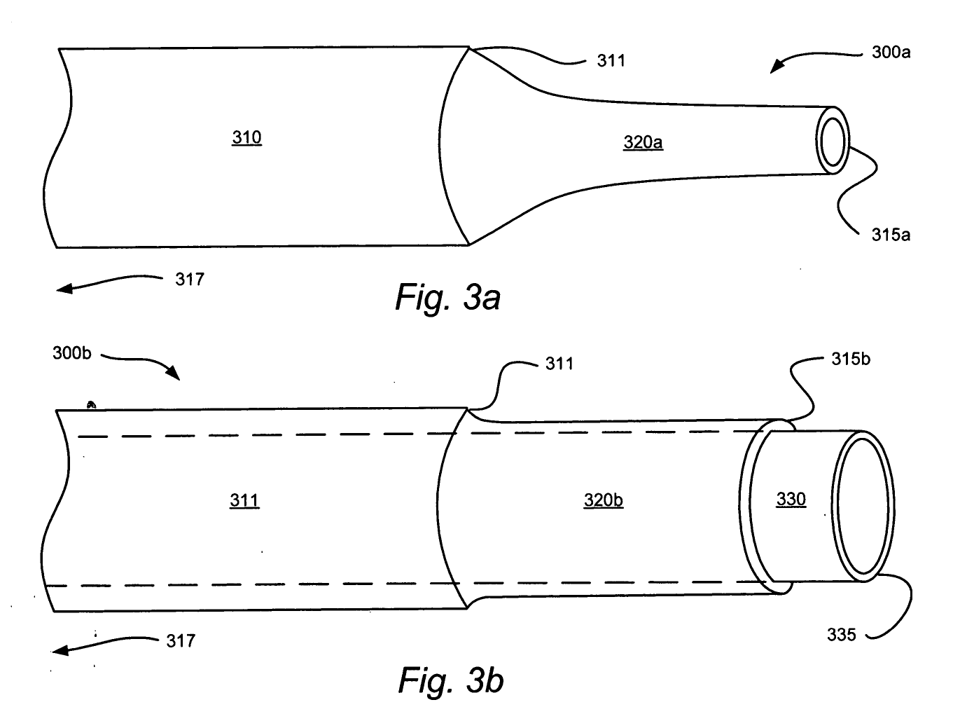

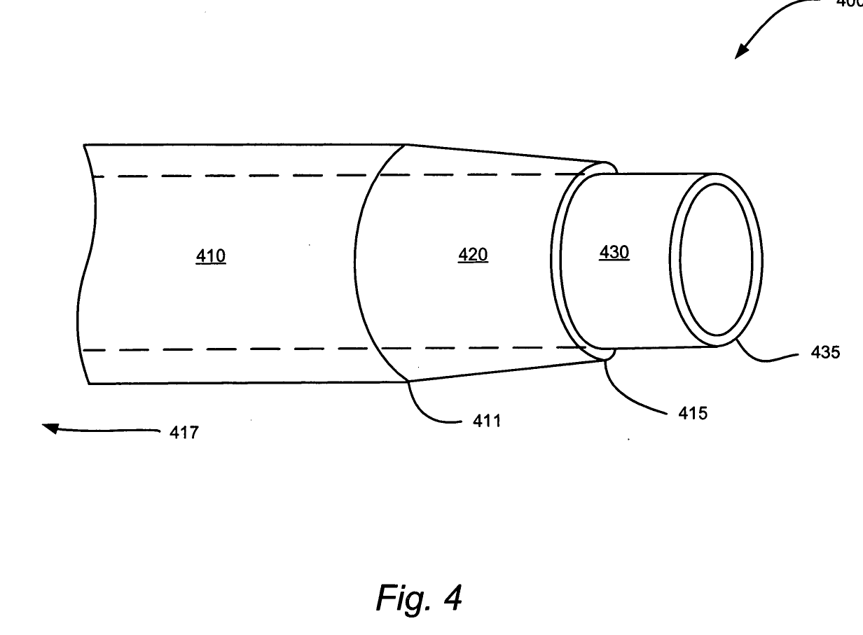

Turning to FIGS. 3, an embodiment of a tapered tip catheter sheath 300 in accordance with other embodiments of the present invention is illustrated. As depicted in FIG. 3a, catheter sheath 300 includes a catheter body 310 extending from a proximal end 317 to a catheter tip 320a that terminates in a distal end 315a. The general shape of catheter tip 320 is referred to as a “rounded step taper” where the taper is somewhat rounded with the rounding somewhat more pronounced near a joint 311, and in this particular case it can be referred to as “concave in” due to the inward rounding. Alternatively, the tip can be “concave out” where the rounding turns the opposite direction, or the taper can be “smooth” where the rounding either does not exist or is not pronounced, or where the curve does not increase or decrease substantially near joint 311. In this embodiment, catheter tip 320a is made of a deformable polymer material that is attachable to catheter body 310. In some cases, the deformable material exhibits memory such that catheter tip 320a is capable of expanding when a force is applied, but returns at least somewhat to the shape exhibited prior to deformation. Alternatively, in some cases, the deformable material does not include memory and will assume approximately the deformed shape after the force that caused the deformation is removed.

FIG. 3b illustrates catheter sheath 300 upon insertion of a guide catheter 330. As illustrated, guide catheter 330 is inserted into proximal end 317 of catheter sheath 300 and advanced until a distal end 335 of guide catheter 330 extends through and beyond distal end 315 of catheter sheath 300. Prior to insertion, the outer diameter of guide catheter 330 is greater than distal end 315a. Thus, upon insertion, guide catheter 330 causes tip 320a to deform to exhibit tip 320b. Such an approach can be used to create a catheter distal end that is concave in, and in some cases a catheter distal end that exhibits a smooth transition from distal end 315 to catheter body 310.

In one embodiment of the present invention, catheter sheath 300 is manufactured by providing an elongate cylindrical tube that includes a lumen extending from one end of the tube to the other, and a conical tube with a lumen extending therethrough. A distal end of the tube is attached to a proximal end of the conical tip forming a joint 311. In other embodiments of the present invention, catheter sheath 300 is manufactured by providing the previously described elongate cylindrical tube and another relatively short cylindrical tube. The distal end of the elongate tube is attached to a proximal end of the short cylindrical tube forming joint 311. During the attachment process, heat or other energy can be applied to the short cylindrical tube causing it to contract such that the diameter of the distal end is reduced, while the diameter at joint 311 is maintained by the relatively deformation resistant material used to form the elongate tube. It should be noted that either or both of the elongate tube or the tube forming the tip of catheter sheath 300 can include a number of sections or portions similar to that described in relation to FIG. 2b above. Further, based on the disclosure provided herein, one of ordinary skill in the art will appreciate a number of other methods and/or materials that can be used in manufacturing catheter sheath 300 that will fall within the scope of one or more embodiments of the present invention.

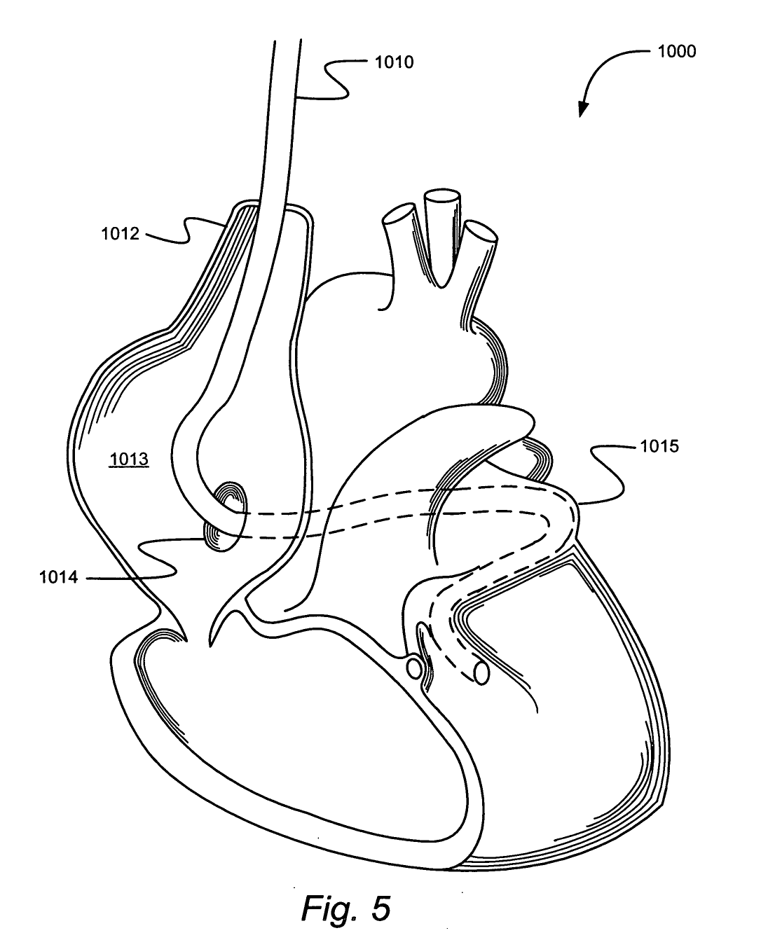

Turning to FIG. 4, a smooth tapered catheter sheath 400 in accordance with other embodiments of the present invention is illustrated. Catheter sheath 400 includes a body 410 extending from a proximal end 417 to a joint 411 representing the distal end, and a tip 420 exhibiting a smooth taper extending from joint 411 to a distal end 415 of catheter sheath 400. A guide catheter 430 extending through and beyond catheter sheath 400 is shown for reference. Such a catheter sheath can be manufactured of materials and using processes previously described herein, and/or other processes or materials known in the art.

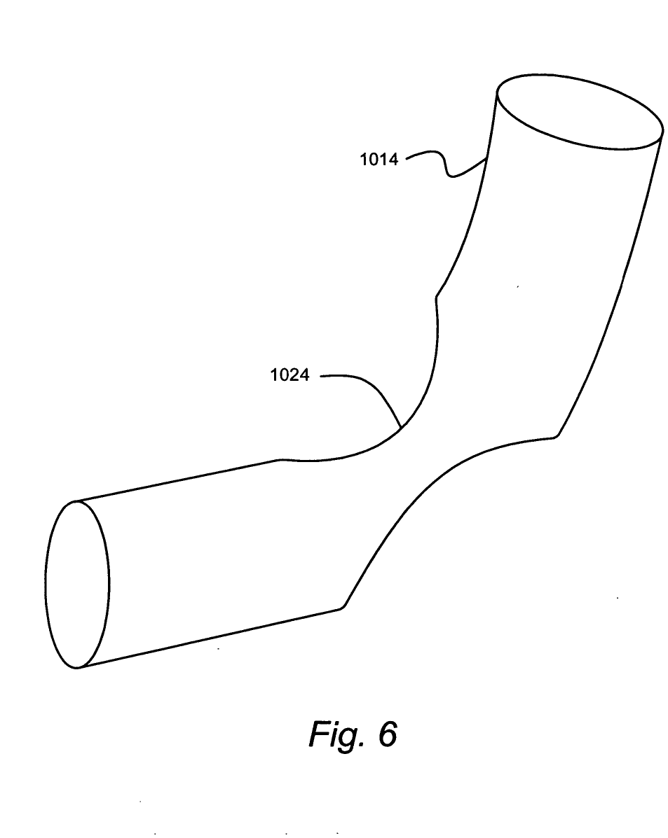

Based on the disclosure provided herein, one of ordinary skill in the art will appreciate that the systems and methods of the present invention can be applied in a number of circumstances. As just one example, embodiments of the present invention can be applied when deploying a lead through and beyond the coronary sinus (“CS”) of a heart. With reference to a heart 1000 depicted in FIG. 5, a typical lead deployment includes directing a nested catheter 1010 through the superior vena cava 1012, the right atrium 1013, the CS 1014, and into the great vein 1015 of heart 1000. As can be appreciated from FIG. 5, traversing through the heart involves a number of turns that can be difficult in most patients, and very difficult in patients with abnormal anatomy such as those suffering from heart failure. To aid in traversing through the heart, the guide catheter of nested catheter 1010 exhibits a different shape than that of the outer catheter sheath. Because of the variation in shape, when the guide catheter is moved relative to the outer catheter sheath, the direction assumed by the nested catheter tip can be controlled.

In a typical lead deployment, nested catheter 1010 is advanced through superior vena cava 1012 into right atrium 1013. At this point, the eustachian ridge protruding into the right atrium from the posterior wall thereof; the coronary sinus OS located behind the eustachian ridge; and the thebesian valve interfere with entering into CS 1014, and can also be relied upon to guide entry into CS 1014. To this end, the tip of nested catheter 1010 is disposed along the superior, posterior wall of right atrium 1013 where advantage can be taken of the eustachian ridge and the thebesian valve to direct the tip into the coronary sinus OS. With the catheter in this location, the guide catheter can be advanced such that it extends beyond the end of the outer catheter. In addition, a counterclockwise torque can be applied to the guide catheter causing the tip thereof to advance until it touches a feature of the heart. Continued counterclockwise torque causes the tip of the guide catheter to follow the feature(s) of the heart until the tip is positioned in the opening of CS 1014. With nested catheter 1010 in this position, the guide catheter is advanced into CS 1014, and the outer catheter sheath is then advanced over the guide catheter and into CS 1014.

It has been found that difficulty can occur when attempting to advance the outer catheter sheath into CS 1014. Using an intermediate catheter sheath in accordance with one or more embodiments of the present invention can reduce, and in some cases eliminate this difficulty. For example, an intermediate catheter sheath as discussed in relation to FIGS. 2 above can aid in entering CS 1014. In particular, it may be possible to advance the intermediate catheter sheath over the guide catheter and into CS 1014. This can gently expand the opening of CS 1014, and consequently allow the outer catheter sheath to advance over the intermediate catheter sheath and into CS1014. As another example, the outer catheter sheath can include a tapered tip as discussed in relation to FIGS. 3 and 4 above. Such a tapered tip has less tendency to hang up the opening of CS 1014 when compared to a non-tapered tip, and thus can be effectively used as the outer catheter sheath advances over the guide catheter and into CS 1014. It should be noted that the preceding discloses only one application for various embodiments of the present invention, and that one of ordinary skill in the art will recognize a number of other applications of the present invention.

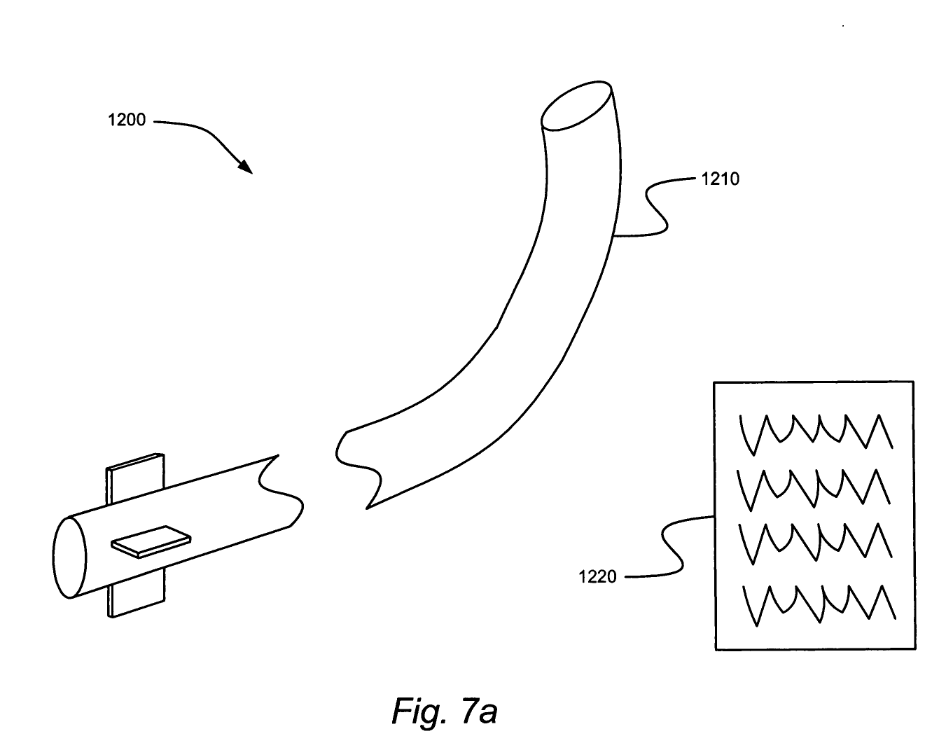

Turning to FIG. 6, another example applying one or more embodiments of the present invention is discussed. In the example, CS 1014 has become restricted by what can be referred to as a “coronary sinus spasm”. In particular, an area 1100 tightens for one of a variety of reasons including, for example, stimulation by a guide catheter passing through CS 1014. In such a case, an outer catheter sheath may not be capable of passing through area 1100 without injuring CS 1014. To avoid an injury, a tapered end catheter sheath in accordance with the present invention may be useful to gently expand area 1100. This can be done by a single catheter sheath having a tapered end and/or by an intermediate catheter sheath of smaller diameter than the outer catheter sheath and disposed over the guide catheter.

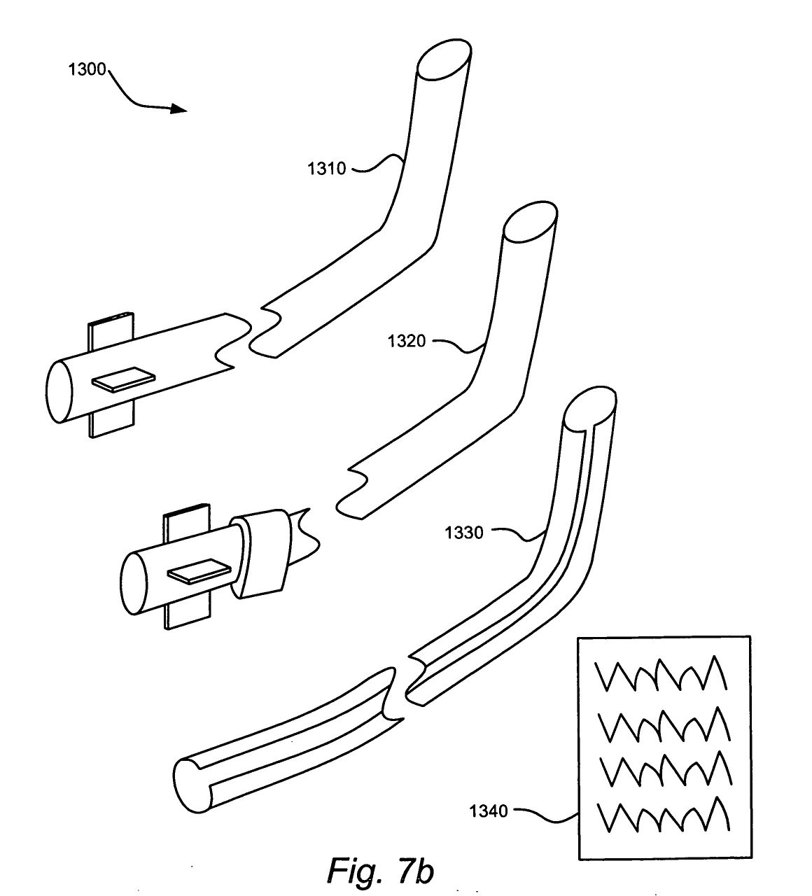

Turning to FIGS. 7, two exemplary catheter kits 1200, 1300 are discussed. As depicted in FIG. 7a, catheter kit 1200 includes an intermediate catheter sheath 1210 with some instructions 1220. In some cases, instructions 1220 include an indication of catheter sizes to which intermediate catheter sheath 1210 can be applied. In one particular case, instructions 1220 indicate that intermediate catheter sheath 1210 can be used with a guide catheter with an outer diameter of 2.06 millimeters or less. Further, instructions 1220 can indicate that intermediate catheter sheath 1210 can be used with an outer catheter sheath with an inner diameter of 2.21 millimeters or more. Instructions 1220 indicating other measurements including, but not limited to those disclosed herein can also be included with the appropriate intermediate catheter sheath. Additionally, instructions 1220 can indicate material compatibility between intermediate catheter sheath 1210 and other catheter sheaths and/or guide catheters. Yet further, instructions 1220 can indicate model numbers of guide catheters and/or outer catheter sheaths with which intermediate catheter sheath 1210 can be used. It should be recognized that intermediate catheter sheath 1210 can be similar to one or more embodiments of the present invention as previously described.

As depicted in FIG. 7b, catheter kit 1300 includes an outer catheter sheath 1310, a guide catheter 1320, and an intermediate catheter sheath 1330. Further, catheter kit 1300 includes some instructions 1340 explaining tolerances of the various kit components and/or use of the kit components. It should be noted that while intermediate catheter sheath 1330 is shown with a slit body similar to that of FIG. 2c, non-slit sheaths in accordance with other embodiments of the present invention can also be included in kit 1300. Based on the disclosure provided herein, one of ordinary skill in the art will recognize one or more additional kit elements that can be included in kits 1200, 1300 according to embodiments of the present invention. Further, based on the disclosure provided herein, one of ordinary skill in the art will recognize one or more elements that can replace corresponding elements of kits 1200, 1300 according to other embodiments of the present invention.

In conclusion, the present invention provides novel systems, methods and arrangements for catheterization. While detailed descriptions of one or more embodiments of the invention have been given above, various alternatives, modifications, and equivalents will be apparent to those skilled in the art without varying from the spirit of the invention. Therefore, the above description should not be taken as limiting the scope of the invention, which is defined by the appended claims.

1. A catheter sheath for forming a tapered distal end of a catheter system, wherein the catheter sheath comprises:
a first elongate tube, wherein the first elongate tube includes a proximal end, a first distal end, and a lumen extending between the proximal end and the first distal end;
wherein the first elongate tube is disposable over a guide catheter; and
wherein the first elongate tube is disposable within a second elongate tube having a second distal end such that the first distal end extends through and beyond the second distal end forming a stepped taper.

2. The catheter system of claim 1 wherein the first elongate tube further includes an opening extending from the proximal end to the first distal end, wherein the opening allows for wrapping the first elongate tube around an element previously disposed within the second elongate tube.

3. The catheter system of claim 1 wherein the distal end of the first elongate tube is angled.

4. The catheter system of claim 1 wherein the first elongate tube further comprising a plurality of electrodes.

5. The catheter system of claim 1 wherein the distal end is tapered such that an outside diameter of the distal end decreases in a distal direction.

6. The catheter system of claim 1 wherein the at least one elongate tube includes an outer elongate tube and an inner elongate tube; wherein the outer elongate tube includes an outer proximal end, an outer distal end, and an outer lumen extending between the outer proximal end and the outer distal end; wherein the inner elongate tube includes an inner proximal end, an inner distal end, and an inner lumen extending between the inner proximal end and the inner distal end; wherein the inner distal end extends through and beyond the outer distal end forming a stepped taper.

7. The catheter system of claim 6 wherein the inner elongate tube further includes an opening extending from the inner proximal end to the inner distal end, and wherein the opening allows for wrapping the inner elongate tube around an element previously disposed within the outer elongate tube.

8. The catheter system of claim 5 wherein the taper is a smooth taper.

9. The catheter system of claim 5 wherein the taper is a rounded step taper.

10. The catheter system of claim 9 wherein the rounded step taper is concave in.

11. The catheter system of claim 1 wherein the distal end is formed of a deformable material.

12. The catheter system of claim 1 wherein the deformable material has memory.

13. The catheter system of claim 1 wherein a guide wire is disposable within the guide lumen.

14. The catheter system of claim 1 wherein the distal end is formed of a first material, and wherein a portion of the elongate tube joining the distal end is formed of a second material.

15. The catheter system of claim 1 wherein a guide is disposable within the lumen, and wherein an outer diameter of the guide is less than the first inner diameter and greater than the second inner diameter when the guide is partially disposed in the lumen.

16. A method for disposing an object in the coronary sinus of a heart, the method comprising:
providing an outer elongate tube including an outer proximal end, an outer distal end, and an outer tube lumen extending from the catheter proximal end to the catheter distal end;
providing an inner elongate tube including an inner proximal end, an inner distal end, and an inner tube lumen extending between the inner proximal end and the inner distal end;
inserting the outer distal end within the right atrium of a heart; and
inserting the inner distal end through the outer lumen and extending beyond the outer distal end, wherein the combination of the outer distal end and the inner distal end forms a stepped taper.

17. The method of claim 16 further comprising inserting a guide into the inner tube, wherein the guide includes a guide distal end and a guide proximal end.

18. The method of claim 17 wherein the guide includes a lumen extending between the guide proximal end and the guide distal end, and wherein the method further comprises:
inserting the guide distal end into the heart, wherein the guide distal end contacts the coronary sinus; and
sliding the catheter sheath over the guide, wherein the catheter distal end contacts the coronary sinus; and
advancing the catheter distal end into the coronary sinus.

19. The method of claim 16 further comprising advancing a lead through the inner tube.
